(19) United States
(12) Patent Application Publication
(10) Pub. No.: US 2018/0064853 A1
(43) Pub. Date: Mar. 8, 2018

(54) Hierarchically Structured and Multifunctional Nanofibrous Composite Structure for Soft-Tissue Engineering Applications

(71) Applicants: Roohollah Bagherzadeh, Tehran (IR); Masoud Latifi, Tehran (IR); Lingxue Kong, Waum Ponds (AU)

(72) Inventors: Roohollah Bagherzadeh, Tehran (IR); Masoud Latifi, Tehran (IR); Lingxue Kong, Waum Ponds (AU)

(21) Appl. No.: 15/256,741

(22) Filed: Sep. 6, 2016

(51) Int. Cl. A61L 27/48; C12N 5/00

(52) U.S. Cl. CPC A61L 27/48; C12N 5/0068; A61L 2400/12; C12N 2537/00; A61L 2430/34; C12N 2533/30

(57) ABSTRACT

The disclosed method presents a method of fabrication of wrapped multi-scale nano-to-micro fibrous structures near-similar to extracellular matrix (ECM) structure. The resulting materials finely mingle nano-scale fibers on micro-scale fibers to form a composite structure with defined responsibility of each fiber category for diffident application including soft-tissue engineering. This composite-like structure of the fibrous material may be helpful in cell differential regulation when different cell types are necessary in a tissue. This hierarchically structure of nanofibers, as a cell-adhesive matrix, on the micro-scale fibers, as an elastomeric structural component, present a favorable structure most similar to the natural ECM and therefor acts as a growth factors for recruitment and cell proliferation within the structure.

### BACKGROUND OF INVENTION

The design and fabrication of biocompatible scaffolds for tissue regeneration is a multidisciplinary study. Biodegradable scaffolds should provide selected cell cultures with a suitable substrate for adhesion, proliferation, and produce their own extracellular matrix (ECM) (Soliman, Pagliari et al. 2010). Electrospun fibrous scaffolds have received great interest in recent years due to their promising properties which make them suitable structure for mimicking the ECMs (Sisson, Zhang et al. 2010, Bagherzadeh, Latif et al. 2014).

Electrospun fibrous mats have been investigated as scaffolds to engineer various tissues such as bone (Sisson, Zhang et al. 2010), vascular scaffolding system (Marelli, Alessandrino et al. 2010), cardiac tissue (Shin, Ishii et al. 2004), peripheral nerve system (Ghasemi-Mobarakeh, Prabhakaran et al. 2011), ligament/tendon and intervertebral disc (Makris, Hadidi et al. 2011), and skin (Lowery, Datta et al. 2010). There are a few basic requirements that have been widely accepted for fibrous scaffolds such as biocompatibility, reliable thermal and mechanical properties similar to those of ECMs, adequate surface properties, and appropriate three-dimensional (3D) pore architectures (Blakeney, Tambralli et al. 2011, Rnjak-Kovacina and Weiss 2011).

It is believed that Pore size in a fibrous scaffold affects cell binding, migration, cellular in-growth, and phenotypic expression. Many studies (Rezwana, Chena et al. 2006, Orlova, Magome et al. 2011) have been also performed to understand the interaction between cells and scaffolds made of different both natural and synthetic polymers including chitosan, alginate, starch, collagen and gelatin, for natural polymers, and polyesters, polyorthoesters, polyacetals, poly(α-amino acids), poly(cyanoacrylates), and poly(anhydrides), for the class of synthetic polymers.

Natural polymers, in spite of their proven biocompatibility, are often associated with inherent limitations such as poor mechanical strength and uncontrolled degradation. Synthetic polymers, on the other hand, can provide reliable mechanical properties and degradation characteristics for a variety of tissue engineering applications; however, their hydrophobicity causes poor wettability, lack of cell attachment and uncontrolled biological interactions with the material (Sill and von Recum 2008). So, each polymer system has a weakness in biological regulation or mechanical requirement, and therefore many researchers have been explored blended synthetic polymers with natural polymers (Kluger, Wyrwa et al. 2010).

Some studies have been also focused on altering the nozzle and collector configurations to produce scaffold with different fiber diameter (Badami, Krekea et al. 2006, Hong and Madihally 2011). In their studies, it has been revealed that cell adhesion with Nano-size fibers is better than Micro-size fibers, whereas some show the contrary results (Badami, Krekea et al. 2006). On the other hand, fibers with larger diameter increase the pore size in the electrospun scaffolds, leading to increase cell infiltration (Rnjak-Kovacina, Wise et al. 2011, Bagherzadeh, Latif et al. 2014).

For instance, Human mesenchymal stem cells showed better chondrogenesis on Micro-sized fiber compared to Nano-size fibers due to their large pore size, but rat neural progenitor cells showed improved proliferation in the nervous system on Nano-fibers compared to Micro-fibers (Christopherson, Song et al. 2009). Scaffold mechanical properties also play an important role in tissue regeneration and in particular, morphogenesis of cells. Both the micro and nano-scale fibers in scaffold are important during tissue regeneration. At the micro-scale, cellular activity is shown to be influenced by the substrate stiffness and also physical support for both the scaffold and the surrounding tissue can be provided (Zaleskas, Kinner et al. 2001, Sieminski, Hebbel et al. 2004).

Fibers with nano-scale also plays an important role in distributing stress forces uniformly in tissues (Ushiki 2002) and have more-specific surface area, thereby offering a larger number of available focal adhesion points for cell attachment (FIG. 1).

It has been revealed that the natural ECM of the body consists of fibrous and soluble proteins as well as other bioactive molecules with a three-dimensional topography (Kluger, Wyrwa et al. 2010, Votteler, Kluger et al. 2010). It is believed that fibrous components of the ECMs form a composite-like structure and they are divided into three types of fibers: collagen, reticular and elastic. Collagen fibrils are cylindrical in shape with mean diameter about 40-80 nm (Kidoaki, Kwon et al. 2005).

Collagen fibrils in the reticular fibers have also rather thin and uniform diameter, ranging from 20-40 nm. On the other hand, Elastic fibers and laminae are composed of micro-fibrils and elastin components (Ushiki 2002). The study of fibers arrangement in the ECM is important for understanding the functional role of the fibers in tissue and more importantly, for engineering a scaffold structure for tissue regeneration. On the other hand, elastin fibers, with diameter about 0.2-1.5 μm, form a loose and coarse network which it is believed that their organization with laminae influences the resilience of tissues for their mechanical properties.

Therefore, bio-mimicking similar features of ECM (FIG. 1) have led to the development of ordered Nano-scale scaffolds in the same dimensional scale. Also, nano-dimensional surface features enhanced cell adhesion and proliferation better than micro-dimensional surfaces of the same material. But producing the nanofiber with ranging diameter less than 100 nanometer usually have some defect like beads on them and it is well documented that beaded scaffolds offer the lowest cell adhesion and minimal growth kinetics (Badami, Kreke et al. 2006).

### SUMMARY OF INVENTION

This invention presents a novel deposition which allows wrapping nanofibers on the microfibers within the fibrous structures composed of two polymers with having control on their architecture. Since, different polymers are present in different part of layers, as well as single fibers, total mechanical property is quarantined by the interfacial strength of the micro-scale fibers, and cell attachment and growth factors are provided by the nano-scale fibers. So by having this structure, one can have a fibrous structure with a nanofibers wrapped on the microfibers in a one-step process of fabrication (See FIG. 2).

According to one aspect, a favorable structure for tissue regeneration application, may be most similar to the natural extracellular matrix (ECM) composed at least two different scales of nano and micro fibers. This structure of the fibrous material may be helpful in cell differential regulation when different cell types are necessary in a tissue. This hierarchically structure, therefor, acts as a growth factor for recruitment and cell proliferation within the structure.

According to another aspect, a method of making a fibrous structure with the aforementioned specifications should be capable of scaling up, simple, affordable, and having control of making the structures with pre-defined specifications.

As a best mode at the present time, a method of making a fibrous structure with two new electrospinning techniques, multilayering electrospinning and mixing electrospinning, were devised to design a hierarchically ordered structure of the matrices and scaffolds composed of nano- and microscale wrapped fiber intermeshes for tissue-engineering devices. Mesoscopic spatial depositions and structural proposed in this invention may find many other new applications in different section of engineering applications.

### BRIEF DESCRIPTION OF DRAWINGS

FIG. 1 depicts SEM images of natural elastic fibers in the mouse aortic adventitia (Ushiki 2002).

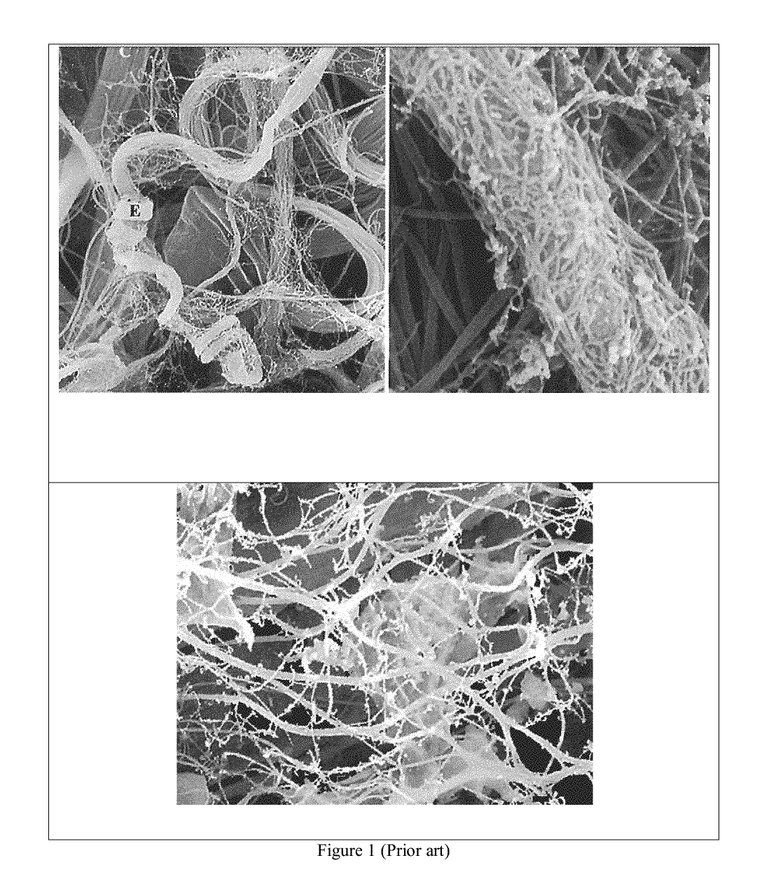

FIG. 2 shows typically representative SEM photographs of fibrous structure of present invention with different hierarchical structure.

### DETAILED DESCRIPTION OF SPECIFICATION

The present invention is directed to hierarchically structured and multifunctional Nanofibrous composite structures that comprise wrapped multi-scale nano-to-micro fibers near-similar to extracellular matrix (ECM) structure.

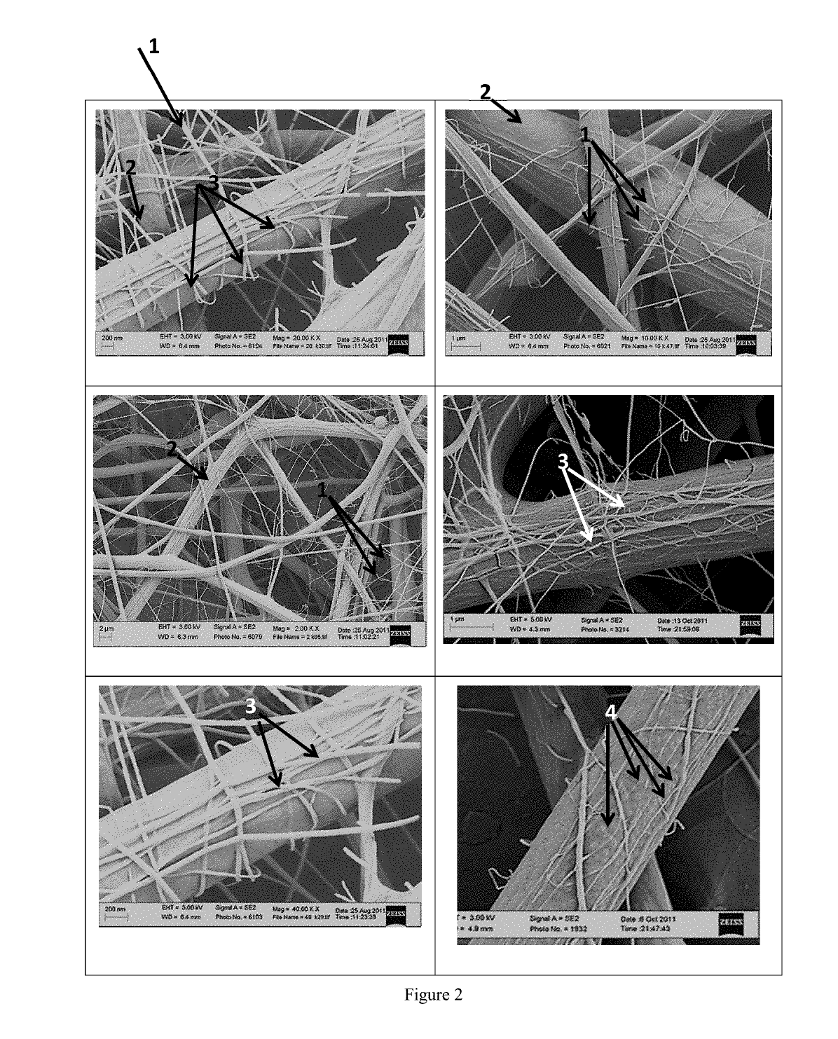

The term “nanofiber” as used herein refers to fibers having a diameter or cross-section between about 5 nanometers (nm) and 50 nm, preferably less than 30 nm (1 in FIG. 2). The term “microfiber” also refers as fibers with more than 100 nm in their diameters or cross sections (2 in FIG. 2).

The term “wrapped fibers” refers to nanofibers wrapped on the microfibes (3 in FIG. 2). These types of fibers have a unique specification which enables one to have interesting specific area on the surface of micro fibers. Therefore mechanical properties of structure are guaranteed by the microfibers and therefore nanofibers can be used for any subject of interest such as a placement for cells in tissue engineering application.

Having the structure of the present invention has many benefits. For example if the structure used in tissue engineering application as a scaffold, it is possible to have a predefine pore structure within fibrous structure of scaffold by controlling the presence of nanofibers (1 in FIG. 2) in the area between the microfibers.

The composite structure of fabricated fibrous material according to the present invention can be additionally useful for many applications since it can be possible to have to polymer type within one fiber (one polymer as a matrix and one polymer as a filled part in the provided matrix, see 4 in FIG. 2).

The material described above is especially suited for use on tissue engineering as a scaffold. For example, nano-dimensional surface features enhanced cell adhesion and proliferation better than micro-dimensional surfaces of the same material. But producing the nanofiber with ranging diameter less than 100 nanometer usually have some defect like beads on them and it is well documented that beaded scaffolds offer the lowest cell adhesion and minimal growth kinetics (Badami, Kreke et al. 2006).

So, having a composite nanofibrous scaffold contains both nano-scale (20-100 nm) and micro-scale (0.2-2 μm) fibers, the smaller fiber diameter of scaffold provide a larger surface area-to-volume ratio to bind more cell growth factors. Smaller diameter fibers are more flexible and pliable than larger diameter fibers. Therefore, cells require less force to migrate within and over the small diameter fibers than fibers with micro-scale in diameter. Further, required structural mechanical properties of the scaffolds are provided by bigger fibers in the scaffolds. Therefore, generating structures with suitable and controllable Micro/Nano-architecture is still much favorable for researchers.

Using this strategy (having nano and micro scale fibers within the scaffold), the inherent advantages of both Nano-fibers and Micro-fibers can be realized in a single scaffold. Up to now, researchers have utilized multi-layering techniques to construct a bimodal scaffold consisting of alternating layers of micro and nanofibers (Karageorgiou and Kaplan 2005, Kidoaki, Kwon et al. 2005, Soliman, Pagliari et al. 2010). Basically, two different polymers are simultaneously electrospun from different syringes under special conditions.

The produced fibers are mixed on the same collector, resulting in the formation of a mixed fiber mesh (Kidoaki, Kwon et al. 2005, Pham, Sharma et al. 2006). Although applying these techniques, one can produce a scaffold with two (or more) fiber diameter distributions; there are still a few big limitations in tissue engineering applications including: 1) the deposited layers are not exhibit significant adhesion to the other layers; 2) importantly no entanglement of two different fibers occurs in principle; 3) sequential layering of the different polymer on the collector, reducing the conductivity of the collector happened and make this technique limited only for thin layered scaffold; and 4) homogeneity of the structure is difficult to achieve due to the increasing electrostatic repulsion amongst the accumulating fibers as the scaffold thickness increased (Kidoaki, Kwon et al. 2005, Kluger, Soliman, Pagliari et al. 2010, Hong and Madihally 2011).

Therefore, by the structure presented in this invention the advantages of two biocompatible polymers, for example PCL and gelatin, can be combined well. So by having this structure, one can have a high surface area for cell attachments as well as low mechanical and thermal strength for infiltration into multilayer of scaffolds. Moreover, component percentage of composite (or the percentage controllability of nano and micro fibers in the form of wrapped fibers) would be of great significance to the biomaterial field for different cells and cultures.

### Test Methods and Fabrication Process

To obtain the structures presented in this invention, biodegradable a one-step fabrication by electrospinning process is utilized. By varying the processing parameter of electrospinning (including the applied voltage and nozzle to collector distance) and solution parameters (polymer concentration and solvent percentage), different shape of hierarchical structure of nanofibers wrapped on the microfibers is achieved. Polycaprolacton and gelatin (type A, from porcine skin) were dissolved separately in 2,2,2-trifluoroethanol (TFE) at different concentration of 12-16% wt/v. Then polymers blend solution of PCL/gelatin with different volumetric ratio 1:1, 1:3, and 3:1 under gentle stirring is prepared for electrospinning. Polymer solution was feed by syringe pump (Fanavaran Nano-Meghyas Company) at different rates of 0.5 to 6 ml/hr through the nozzle. A voltage of 12-20 KV was applied to the nozzle with a high voltage power supply. A set of collectors was placed with needle-tip to collector distance of 12-20 cm. The morphologies of nanofiber scaffolds were characterized by scanning electron microscope (SEM).

1. A method for producing a hierarchically structured and manufactured nanofibrous composite structure for soft tissue engineering application comprising the steps of: dissolving two different biocompatible polymers separately in a solvent at different concentration of 12-16% wt/v, creating a polymer blend solution; wherein said polymer blend solution with different volumetric ratio under gentle stirring is prepared for electrospinning; said polymer solution is then fed by a syringe pump at different rates through a nozzle, where a voltage is then applied to said nozzle via a high voltage power; a set of collectors was placed with a predetermined needle-tip to collector distance; creating a nanofibrous scaffold containing both nano-scale and micro-scale fibers, wherein smaller fiber diameter of said scaffold provides a larger surface area-to-volume ratio to bind more cell growth factors; wherein said smaller diameter fibers are more flexible and pliable than larger diameter fibers; therefore cells require less force to migrate within and over said smaller diameter fibers than fibers with said micro-scale fibers.

2. The method of claim 1, wherein two new electrospinning techniques are a multilayering and mixing electrospinning.

3. The method of claim 2, wherein said two biocompatible polymers comprising PLC, Polycaprolacton and gelatin (type A); and wherein said solvent comprises 2,2,2-trifluoroethanol (TFE).

4. The method of claim 3, wherein by varying processing parameter of electrospinning, including an applied voltage and nozzle to collector distance and solution parameters including polymer concentration and solvent percentage, different shape and hierarchical structure of nanofibers wrapped on microfibers is achieved.

5. The method of claim 4, wherein said polymer solution having a high surface area for cell attachment as well as low mechanical and thermal strength for infiltration into multilayer of scaffolds

6. The method of claim 5, wherein said different rate comprises a range of 0.5 to 6 ml/hr, and said voltage comprises a range of 12-20 KV.

7. The method of claim 6, wherein said predetermined distance is 12-20 cm and wherein said volumetric ratio comprises a range of 1:1, 1:3 and 3:1.

8. The method of claim 7, wherein said method is a one-step fabrication process of wrapped nanofibers on microfibers within a fibrous structure, wherein a formation of said nanofibers on said microfibers can be changed with process and solution parameters in a typical electrospinning process.

9. The method of claim 8, wherein said wrapped nanofibers can be slightly different polymer component (gelatin) compared to said microfibers (gelatin in PCL matrix).

10. The method of claim 9, wherein different portion of one of said two polymers can be found in a matrix of said microfibers to form a composite structure within a single microfiber.

11. The method of claim 10, wherein said diameter or cross-section of said nanofibers is ranged from 10 to 50 nm, and for said microfibers are in micrometer scale.

12. The method of claim 11, wherein said hierarchically structure presents a favorable structure most similar to a natural ECM and therefor can be a suitable fibrous scaffold for tissue engineering application.
